(19) United States
(12) Patent Application Publication
(10) Pub. No.: US 2005/0076398 A1
(43) Pub. Date: Apr. 7, 2005

(54) Animal model of chronic stress diseases and method for producing the same

(75) Inventors: Kazuyoshi Takahashi, Kawasaki-shi (JP); Miroslav Smriga, Kawasaki-shi (JP); Masaki Hashimoto, Kawasaki-shi (JP); Tetsuo Yano, Kawasaki-shi (JP); Hisayuki Uneyama, Kawasaki-shi (JP); Kunio Torii, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Ajinomoto Co., Inc., Tokyo (JP)

(21) Appl. No.: 10/998,832

(22) Filed: Nov. 30, 2004

Related U.S. Application Data
(63) Continuation of application No. PCT/JP03/06846, filed on May 30, 2003.

(30) Foreign Application Priority Data
May 31, 2002 (JP) 2002-160189

(51) Int. Cl. A61K 49/00; A01K 67/027

(52) U.S. Cl. 800/14; 424/9.2

(57) ABSTRACT

The present invention provides animal model of chronic stress diseases produced by feeding lysine deficient diet to the animals and a method for producing the animal model. The effect of medicine on chronic stress diseases can be judged with the animal model. Effective compounds in preventing chronic stress diseases and/or treating patients suffering from chronic stress diseases can be screened by using the animal model.

### BACKGROUND OF THE INVENTION

The present invention relates to animal model of chronic stress diseases produced by feeding lysine deficient diet, a method for producing the animal model, a method for determining the effect of a medicine for patients with chronic stress diseases with the animal model, a screening method for developing medicines for preventing and/or treating chronic stress diseases, and compounds found by the screening.

Recently, the improvement in the quality of life (QOL) is demanded in the medical facilities. An excessive mental stress lowers not only QOL after illness of each patient or in daily life of people but also the social productivity.

As medicines for treating stress diseases, there have been developed various medicines such as anxiolytic agents (such as benzodiazepine derivatives) and antidepressants (such as monoamine uptake inhibitors and tricyclic medicines) used for the purpose of ameliorating the psychologic stressors and also medicines for the treatment of abnormal gastrointestinal motility or visceral pain.

Recently, such life improving medicines and preventive medicines for improving QOL have been expected and many companies have tried to develop them. However, safe and effective therapeutic agents have never been developed yet. In animal model of stress diseases, those of functional gastrointestinal diseases include restraint stress models (K. Miyata et al., J. Pharmacol. Exp. Ther. 261, 297-303, 1992, G. J. Sanger et al., Br. J. Pharmacol. 130, 706-712, 2000) and wrap restraint stress models (C. L. Williams et al., Gastroenterology, 94, 611-621, 1988, C. Ito et al., J. Pharmacol. Exp. Ther. 280, 67-72, 1997). However, those models are unsatisfactory for the models for searching for the effective therapeutic agents in stress diseases which are chronic and psychogenic because the crisis of those models is caused by applying an acute stress to their bodies. Under the circumstances, the existing therapeutic agents (Alosetron, Pinaverium, etc.) for irritable bowel syndrome which is one of the functional digestive tract diseases were mainly evaluated by in vitro tests, and test reports wherein animal models were used were only small in number.

On the other hand, lysine is one of essential amino acids and it is reported that a lack or an insufficient intake of lysine causes mental and physical disorders (D. A. Levitsky and B. J. Strupp, J. Nutr. 125, 2212S-2220S, 1995, D. A. Levitsky and R. H. Barnes, Nature 225, 468-469, 1970, H. P. Chase et. al., Nature 221, 554-555, 1969, P. H. Abelson, Science 164, 17, 1969). Lysine itself enhances the action of an anxiolytic agent, diazepam in animal model of convulsion (C. Yung-Feng and G. Xue-Min, Neurochem. Res. 20, 931-937, 1995), protects the ischemic brains (G. Hong-Ping and K. Bao-Shan, Life Sciences, 65, 19-25, 1999) and is present in a very high concentration in the brains (Q. R. Smith, J. Nutr. 130, 1016S-1022S, 2000). From those facts, it is supposed to be possible that the lack in lysine or insufficient intake thereof causes a psychic load or, in other words, stressed state. Because symptoms (such as reduction in the body weight) due to deficiency in essential amino acids including lysine are recognized several days after the initiation of the deficiency, the change in the symptoms due to the deficiency is not rapidly caused. However, there has never been a report on the change in the stressed state with time induced by lysine deficient diet and also visceral pains and the action on the digestive tracts after the stress. In addition, it is unknown that chronic stress diseases is induced by the intake of lysine deficient diet.

### DISCLOSURE OF THE INVENTION

An object of the present invention is to provide animal model required for the screening of effective medicines for the purposes of judging the therapeutic effect of the medicines on chronic stress diseases or also for the purpose of developing medicines for preventing the chronic stress diseases and/or treating the patients suffering from these diseases.

Another object of the present invention is to provide a method for judging the effect of a medicine on chronic stress diseases by using the animal model.

In addition, another object of the present invention is to provide a method for screening an effective compound in the prevention of chronic stress diseases and/or the treatment of patients suffering from the chronic stress diseases by using the animal model.

A further object of the present invention is to provide effective compounds in preventing chronic stress diseases and/or treating patients suffering from chronic stress diseases by using the above-described screening method.

After intensive investigations done for the purpose of solving the above-described problems, the inventors have found that pathology similar to that of chronic stress diseases can be realized by feeding lysine deficient diet. The present invention has been completed on the basis of this finding.

Namely, the present invention provides animal model suffering from chronic stress diseases produced by feeding lysine deficient diet to the animals.

The present invention also provides a method for producing animal model suffering from chronic stress diseases by feeding lysine deficient diet to the animals.

The present invention also provides a method for judging the effect of medicine on chronic stress diseases by using the above-described animal model.

The present invention also provides a method for screening an effective compound in preventing chronic stress diseases and/or treating patients suffering from chronic stress diseases by using the animal model.

The present invention also provides effective compounds produced by the above-described screening method in preventing chronic stress diseases and/or treating patients suffering from chronic stress diseases.

### BRIEF DESCRIPTION OF THE DRAWINGS

FIG. 1 is a graph showing the results of the determination of the amount of blood corticosterone, adrenal gland weight/body weight ratio and the amount of blood ACTH in lysine deficient rats.

FIG. 2 is a graph showing the results of the visceral sensation threshold in lysine deficient rats and non-deficient rats.

FIG. 3 is a graph showing the effects of various medicines, used for the treatment of irritable bowel syndrome, on abnormal bowels movements in lysine deficient rats.

### BEST MODE FOR CARRYING OUT THE INVENTION

Animals usable in the present invention are not particularly limited so far as they are stressed with diet. Preferably, the animals are preferably mammals (excluding human beings) such as mice, rats, girbils, guinea pigs, rabbits, pigs, cats, dogs and monkeys. More preferably, the animals are rodents, in particular, male Wistar rats of about 7 weeks old.

The expression “chronic stress diseases” herein is a generic name for diseases caused by being exposed to a chronic stress. They include, for example, functional digestive tract diseases and diseases having pathology similar to that of those diseases (such as anxiety disorders (panic disorder and generalized anxiety disorder), somatoform disorders, dissociative disorders, neurosis such as mood disorder, bulimia, nervous anorexia, sleep disorder and diabetic gastrointestinal disorder) and symptoms of the digestive system after the surgical operation of the abdomen. In particular, the functional digestive tract diseases are preferred. As the functional digestive tract diseases, there are known, for example, irritable bowel syndrome, rumination syndrome, globus syndrome, functional heartburn, functional chest pain of presumed esophageal origin, functional gastrointestinal disorders, functional dysphagia, functional vomiting, dysphagia, aerophagia, functional constipation, functional abdominal bloating, functional abdominal pain syndrome, functional diarrhea, sphincter of Oddi dysfunction, gallbladder dysfunction, levator Ani syndrome, functional fecal incontinence, pelvic floor dyssynergia, proctalgia fugax, functional pediatric disorders (infant regurgitation, infant rumination syndrome, cyclic vomiting syndrome, functional gastrointestinal disorder, irritable bowel syndrome, functional abdominal pain, abdominal migraine, aerophagia, functional diarrhea, infant dyschezia, functional constipation, functional fecal retension, functional non-retentive fecal soiling, etc.). In these disorders, irritable bowel syndrome is particularly preferred.

It is not particularly difficult to confirm that the animal model of the present invention is the animal model of chronic stress diseases. For example, as described in a literature of B. P. Chappell et al. (J. Neuroscience, 6 (10): 2908-2914, 1986), an increase in amount of ACTH and corticosterone in the blood is recognized but hypertrophic suprarenal gland is not recognized in the acute stress animal models. On the other hand, an increase in amount of corticosterone in the blood and hypertrophic suprarenal gland is recognized but an increase in amount of ACTH in the blood is not recognized in the chronic stress animal models.

Lysine deficient diet used in the present invention has a lysine content lower than that of ordinary diets. Concretely, an ordinary diet has a lysine content of 13.4 g/kg. On the other hand, Lysine deficient diet used in the present invention has a lysine content of preferably not higher than 1 g/kg and more preferably not higher than 0.3 g/kg. It is desirable to use a diet of a low lysine content prepared by using wheat gluten having a low lysine content as the main starting material. This diet contains ¼ of an ideal lysine requirement and is capable of keeping an increase in body weight of the rats. Namely, it is preferred to feed the diet in such an amount that the amount of lysine is not larger than ¼ of its ideal requirement.

The animal model of the present invention has the pathology of chronic stress diseases, in particular, the pathology of functional digestive tract diseases. The pathology includes, for example, organic changelessness of the digestive tracts, visceral hyperalgesia (J. E. Richter et al., Dig. Dis. Sci., 31: 131-138, 1986, W. E. Whitehead et al., Gastroenterology, 98: 1187-1192, 1990) and abnormal gastrointestinal motility (J. E. Kellow et al., Gastroenterology, 98: 1208-1218, 1990). It is desirable that the animal model has one, two or all patterns of the pathology. The patterns of the pathology are not limited to them.

Lysine deficient diet used for the preparation of the animal model of chronic stress diseases is that described above. The period of time for feeding lysine deficient diet is not particularly limited. However, the continuous feeding is preferred. It is continuously fed for preferably at least 3 days, more preferably 3 to 10 days.

For judging the effect of medicine on chronic stress diseases, in particular, functional diseases of digestive tracts, with the animal model of the present invention, the pathology of the animal model of the present invention can be employed as the index. For example, the effect of the medicine on visceral hyperalgesia or abnormal gastrointestinal motility is evaluated.

For examining visceral hyperalgesia, various methods for determining the pain reaction of animals with a balloon can be employed. For example, one of the methods is a threshold determination method wherein the abdominal muscle contraction is used as the index. However, the methods are not necessarily limited thereto.

For the determination of abnormal gastrointestinal motility, a method for determining a defecation-increasing reaction is preferred. Various methods usable for the determination of intestinal transit function of animals can be employed for this reaction. For example, there is a method wherein carmine dye is used. However, the method is not limited to this method.

The animal model used in those determination methods may be prepared by the above-described preparation method. The animal model is preferably fasted the day before the evaluation.

The animal model of the present invention can be used for the determination of the effect of a compound on chronic stress diseases, particularly functional diseases of the digestive tracts and also for the screening of medicines for preventing and/or treating chronic stress diseases, particularly functional diseases of the digestive tracts. Concretely, the screening is conducted as follows: A compound is administered to animal model of the present invention by the oral, subcutaneous, intravenous or localized administration and then the influence of the compound on visceral hyperalgesia and/or abnormal gastrointestinal motility is evaluated. Compounds which relieved visceral hyperalgesia or abnormal gastrointestinal motility in the screening are prospective medicines for preventing and/or treating visceral hyperalgesia and abnormal gastrointestinal motility caused by chronic stress diseases, particularly functional diseases of the digestive tracts.

The compounds thus screened to be effective for preventing and/or treating chronic stress diseases are used as they are or in the form of a medical composition prepared by mixing them with known, pharmaceutically acceptable carrier, excipient, etc. They can be given by the oral administration in the form of tablets, capsules, powder, granules, pills, etc. or by the parenteral administration in the form of parenteral solution, syrup, ointment, suppositories, etc. The dose which varies depending on the subject, route of administration, symptoms, etc. is about 0.001 mg/day to 5 g/day, preferably about 0.01 mg/day to 2 g/day. This dose of medicine can be administered once a day or in several portions a day.

Animal model of chronic stress diseases of the present invention is an epoch-making model having a very specific characteristic in that they have symptoms similar to those of chronic stress diseases of human beings. This animal model is useful for the judgment of the effects of medicines on chronic stress diseases of human beings. Thus, according to the present invention, the development of medicines for preventing and/or treating chronic stress diseases is remarkably improved.

The following Examples will further illustrate the present invention in detail.

### EXAMPLE 1

Preparation of Low Lysine Diet and Lysine-Containing Diet:

Low lysine diet and lysine-containing diet each having a composition shown in Table 1 were prepared and used in Examples given below.

TABLE 1

| | Low lysine diet | Lysine-containing diet |
|---|---|---|
| | (component %) | (component %) |
| Corn starch | 20.16 | 19.89 |
| Gulten Mix | 28.07 | 28.07 |
| Pre Mix 45 | 45 | 45 |
| Vitamin E | 0.01 | 0.01 |
| Corn Oil | 5 | 5 |
| L-lysine | 0 | 1.35 |
| L-glutamine | 1.76 | 0.68 |
| Total | 100 | 100 |
| Lysine content | 0.3 g/kg diet or less | 13.4 g/kg diet |

### EXAMPLE 2

Determination of Stress Index in Lysine Deficient Rats:

The low lysine diet (n=18) or lysine-containing diet (normal diet; n=18) was given to rats (Charles River Japan, Inc., male Wistar rats, 5W). After feeding for seven days, blood was taken and then centrifuged (3000 rpm, 15 minutes) to obtain a serum and blood plasma. The amount of ACTH in the blood plasma was determined by RIA solid phase method and the amount of corticosterone in the serum was determined by RIA ammonium sulfate salting out method. At the same time, the adrenal gland was taken from each rat and the ratio of the adrenal gland weight/body weight was determined. The statistical analysis was conducted according to Student's t-test.

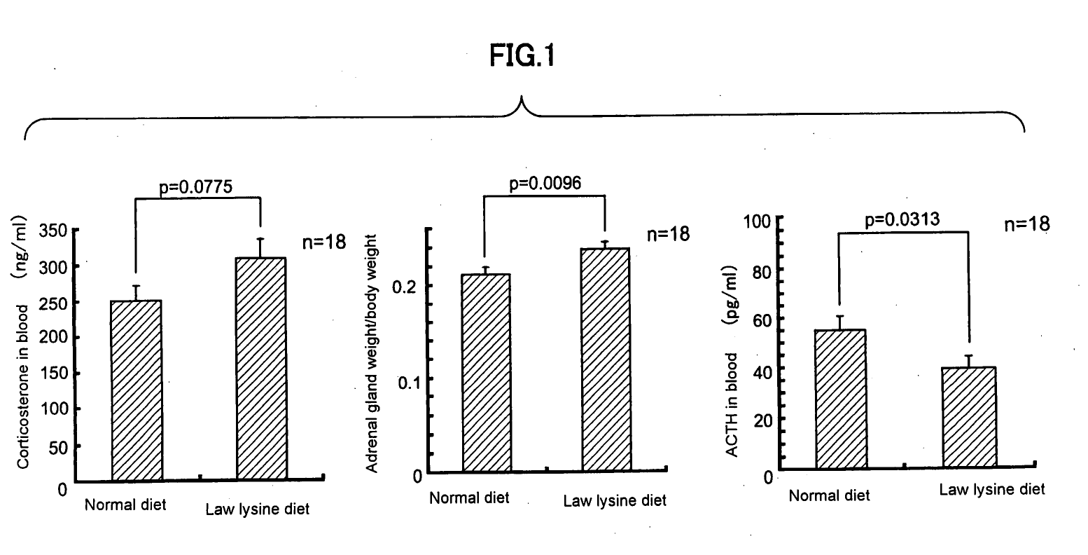

The results are shown in FIG. 1. The rats to which low lysine diet was given for 7 days were inclined to have an increased amount of corticosterone in blood which is well known to be an index caused by the stress exposure, as compared with the rats to which the normal diet was fed. In addition, the rats fed with the low lysine diet for 7 days had an increased ratio of the adrenal gland weight/body weight as compared with that of the rats fed with the normal diet. On the other hand, the blood ACTH which is a factor for controlling corticosterone was decreased in the rats fed with the low lysine diet as compared with that of the rats fed with the normal diet.

### EXAMPLE 3

Organic Change or no Change in the Digestive Tracts of Lysine Deficient Rats:

The low lysine diet (n=3 to 4) or lysine-containing diet (normal diet; n=3 to 4) was given to rats (Charles River Japan, Inc., male Wistar rats, 5W). After feeding for 9 days, the stomach, ileum and colon were extracted from each rat under a light anesthesia with ether and the mucous membrane of each organ was observed with a stereoscopic microscope to determine whether the organic change occured or not and also they were immediately fixed with 4% formaldehyde/10% methanol. These samples were embedded in paraffin and cut into thin pieces. The piece thus prepared was dyed with HE on a slide glass and observed with the microscope.

No unusual change was found in the macroscopic observation and the observation of the HE dyed tissue of the samples of stomach, ileum and colon of the rat fed with the low lysine diet for 9 days like those of the rats fed with the normal diet. In the samples of the rats fed with the low lysine diet, the infiltration of the inflammatory cells and the damage of the mucous membrane were not recognized.

### EXAMPLE 4

Appearance of Visceral Hypersensitivity Induced by Lysine Deficient Rats After Loading the Fasting Stress:

The low lysine diet (n=10) or lysine-containing diet (normal diet; n=8) was given to rats (Charles River Japan, Inc, male Wistar rats, 5W). The rats fed for at least 7 days were used. Each rat was fasted for one day and then a balloon catheter was gently inserted intraanally. A distension balloon was inflated continuously at a rate of 0.9 ml/min by using water until the pressure in the distension balloon had reached 100 mmHg (cut off value). The pressure in the distension balloon, at which the visible contraction of abdominal muscle was detected for the first time, was defined as the visceral sensation threshold. This procedure was done at intervals of 5 minutes. After the visceral sensation threshold was done 5 to 8 times, and then the average of 3 continuous tries as stable values of the threshold was determined as the visceral sensation threshold. The statistical analysis was conducted according to Student's t-test.

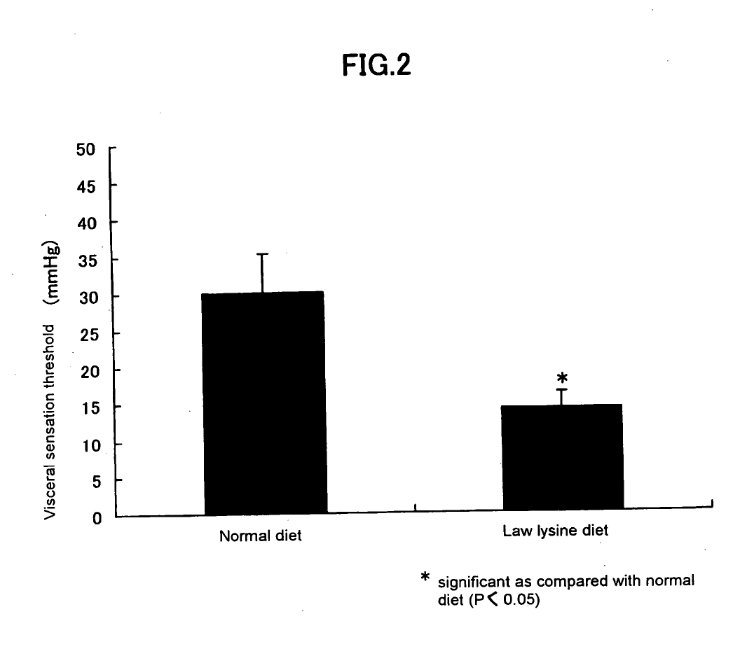

The results are shown in FIG. 2. The visceral sensation threshold of the rats fed with the low lysine diet was 14.23±2.17 mmHg. It was thus confirmed that this visceral sensation threshold was significantly lower than that of the rats fed with the normal diet (29.9±5.37 mmHg).

### EXAMPLE 5

Examination of the Effect of a Medicine on Abnormal Bowel Movement of Lysine Deficient Rats:

The low lysine diet (n=10) or lysine-containing diet (normal diet; n=10) was given to rats (Charles River Japan, Inc, male Wistar rats, 5W). The rats fed for 7 days were used. Each rat was fasted one day before the start of the experiments (on the 6^{th} day after beginning the feeding of the experimental diet) and then a medicine and carmine dye were orally administered. The number of the carmine colorant-containing feces was cumulatively counted sequentially. The statistical analysis was conducted according to Dunnett's method.

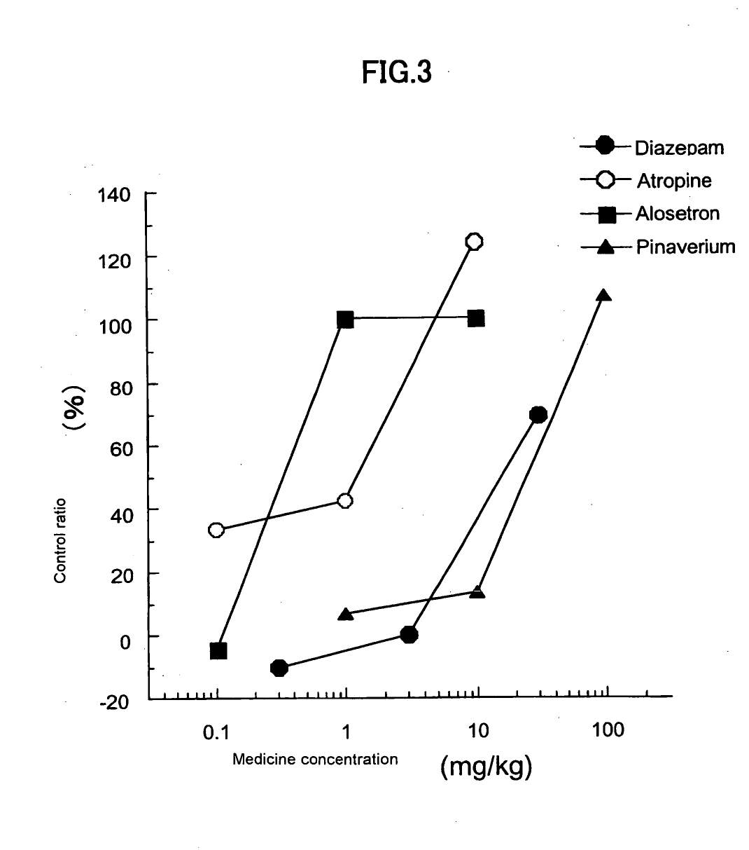

The medicines tested were Diazepam (anxiolytic agent), Atropine (anticholinergic agent) and Alosetron (serotonin 3 receptor antagonist: therapeutic agent for irritable bowel syndrome) and Pinaverium (calcium antagonist of a new type: therapeutic agent for irritable bowel syndrome). The results are shown in FIG. 3. The anxiolytic agent is prescribed for patients suffering from irritable bowel syndrome because the intestinal function is deteriorated by psychologic stress. The anticholinergic agent is sometimes used for the treatment of irritable bowel syndrome because it controls the intestinal movement and secretion and it has an antispasmodic effect. For those models, the anticholinergic agent is used for the clinical treatment of irritable bowel syndrome. All of the four medicines having mechanisms different from one another could significantly control the acceleration of the excretion of the feces of rats in the 7 days after the start of feeding the low lysine diet and they recovered the excretion of feces similar to the excretion of the rats fed with the normal diet.

Thus, the results of the effect of these medicines on the models are similar to those of the medicines on abnormal gastrointestinal motility accompanied with irritable bowel syndrome of human beings. It is understood, therefore, that this model is usable as the animal models of abnormal gastrointestinal motility accompanied with the functional digestive tract diseases including irritable bowel syndrome of human beings and also as the animal model for screening the effect of medicine on the pathology.

The animal model of chronic stress diseases of the present invention is an epoch-making model having no rivals in showing symptoms similar to those of chronic stress diseases of human beings. With the animal models of the present invention, the effect of medicines on chronic stress diseases can be efficiently judged. Namely, according to the present invention, the development of medicines for the treatment and/or prevention of chronic stress diseases are remarkably advanced.

1. Animal model suffering from chronic stress diseases produced by feeding lysine deficient diet to the animals.

2. The animal model of claim 1 which are rodents.

3. The animal model of claim 2 wherein the rodents are rats.

4. The animal model of claim 1, wherein chronic stress diseases are functional digestive tract diseases.

5. The animal model of claim 1, which has at least one symptom selected from the group consisting of organic changelessness of the digestive tracts, visceral hyperalgesia and abnormal gastrointestinal motility.

6. A method for producing animal model suffering from chronic stress diseases by feeding lysine deficient diet to the animals.

7. The method according to claim 6 wherein the lysine deficient diet has a lysine content of not higher than 1 g/kg.

8. The method according to claim 6 wherein the lysine deficient diet has a lysine content of not higher than 0.3 g/kg.

9. The method according to claim 6 wherein the lysine deficient diet is fed to the animals in such an amount that the amount of lysine is not larger than ¼ of the necessary amount.

10. The method according to claim 8 wherein the lysine deficient diet contains gluten as the main ingredient.

11. The method according to claim 6 wherein the lysine deficient diet is continuously fed for at least 3 days.

12. The method according to claim 11 wherein lysine deficient diet is continuously fed for 3 to 10 days.

13. A method for judging the effect of medicine on chronic stress diseases by using the animal model of claim 1.

14. A method for screening a compound effective in preventing chronic stress diseases and/or treating patients suffering from chronic stress diseases by using the animal model of claim 1.

15. Compounds obtained by the method of claim 14 and effective in preventing chronic stress diseases and/or treating patients suffering from chronic stress diseases.
